(19) United States
(12) Patent Application Publication
(10) Pub. No.: US 2026/0266738 A1
(43) Pub. Date: Sep. 10, 2026

(54) METHODS, SYSTEMS, AND APPARATUSES FOR IDENTIFYING, CONTROLLING, AND EVALUATING POLYMER DEGRADATION DURING ELECTRICAL TRACKING TESTS

(71) Applicant: UL LLC, Northbrook, IL (US)

(72) Inventors: Kai-Hsiang Yen, Taipei (TW); Ke-Chia Chen, New Taipei (TW); Chiung-Min Huang, New Taipei City (TW); Hui-Ching Chen, Taipei (TW); Meng-Jie Tsai, Taipei (TW); Kai-Iu Wang, Taipei (TW); Yi-Yiing Chiang, Taipei (TW); Hung-Ping Wu, Taipei (TW); Chung-Han Wu, New Taipei (TW); Dennis Schwarz, Krefeld (DE); Hagen Fasel, Landgraaf (NL); Daniel O'Shea, Melville, NY (US); Ryan Houle, Northbrook, IL (US); Mario A. Xerri, Melville, NY (US); Susumu Arimori, Tochigi (JP)

(21) Appl. No.: 19/181,072

(22) Filed: Apr. 16, 2025

Related U.S. Application Data
(60) Provisional application No. 63/634,766, filed on Apr. 16, 2024.

(51) Int. Cl. G01N 21/88; G06T 7/60

(52) U.S. Cl. CPC G01N 21/8851; G06T 7/60

(57) ABSTRACT

Embodiments for identifying, characterizing, and controlling polymer degradation processes, particularly during electrical tracking tests and accelerated polymer aging evaluations. The embodiments may utilize digital imaging and related technologies coupled with real-time signal correlation to spatially, spectrally, and temporally characterize polymer discharge events and dry-band formation, enabling detailed diagnosis of polymer degradation stages and failure mechanisms. The embodiments further include dynamic feedback control of polymer test parameters based on comparison against stored reference degradation profiles, thus facilitating consistent and repeatable outcomes in accelerated degradation assessments. Additionally, the embodiments an apparatus comprising a controlled-environment chamber, inert gas purging capabilities, pressure regulation systems, and electrodes coated with high-dielectric constant materials, configured to significantly reduce or eliminate air arcing and partial discharge events at elevated test voltages.

### CROSS-REFERENCE TO RELATED APPLICATIONS

The application claims priority to and the benefit of the filing date of provisional U.S. Patent Application No. 63/634,766 entitled “Improvements in Comparative Tracking Index Testing,” filed on Apr. 16, 2024. The entire content of the provisional application is hereby expressly incorporated herein by reference.

### FIELD

The present disclosure relates generally to polymer degradation testing, and more particularly to methods and systems for detecting, characterizing, and controlling degradation processes and failure mechanisms during comparative tracking index (CTI) and electrical tracking tests.

### BACKGROUND

Carbon tracking is a degradation phenomenon in which an electric field applied across a normally insulating polymer material, in the presence of contamination, can initiate localized conductive paths that may grow into an electrical short, potentially resulting in fire. Underwriters Laboratories (UL), as set forth in UL 746A, Section 23, refers to a standardized carbon tracking test for evaluating material resistance to this failure mechanism. This test involves the application of an alternating current (AC) voltage across two platinum electrodes spaced four (4) mm apart on the surface of a conditioned test specimen. A total up to one hundred (100) droplets of a standardized electrolyte solution are applied to the specimen at 30-second intervals, while the applied voltage remains constant. A failure is recorded if the leakage current exceeds a defined threshold, such as 0.5 or 1.0 Amperes, as specified in standards like IEC 60112 or ASTM D3638.

While these tests provide a basic classification of materials according to their tracking resistance, current methods are insufficient for real-time identification and spatial localization of the degradation processes occurring during the test. Carbon tracking is a complex, dynamic phenomenon that involves several concurrent physical and chemical processes—beginning with surface wetting and contamination, followed by the formation of dry bands via Joule heating. This is succeeded by the inception of violet-hued corona discharges across the dry bands, which persist in fixed positions until the contaminant is fully evaporated. As degradation progresses, surface resistance declines due to carbonization, leading to more frequent and mobile scintillation discharges—typically red or orange in color. Ultimately, the leakage current increases as these discharges intensify and propagate, marking the final stages of material failure.

One key limitation of existing techniques is the inability to correlate the evolution of the failure mechanism from inception to completion. During the course of testing, the dominant mechanism can shift—from tracking to erosion to outright combustion—based on a complex interplay of variables such as applied pressure, electrical strength, heat dissipation, material composition, and the presence of additives. Because of this variability, repeatability and reproducibility are poor not only between different laboratories but even within the same laboratory. Without a controlled and traceable progression of failure mechanisms, it is challenging to generate consistent, interpretable test results.

Another challenge arises when test voltages exceed 600 volts. At such elevated levels, air arcing or partial discharge between electrodes becomes sustained, drastically altering the nature of the degradation mechanism. The inception of air arcing is influenced by the generation of free electrons, which are accelerated by the electric field and initiate discharge events through collisions with neutral air molecules. As voltage increases, these collisions become more energetic and frequent, producing more electrons and sustaining the discharge. This introduces a failure mode—air breakdown—that differs significantly from the intended surface tracking evaluation and compromises the relevance of the test for assessing polymer performance in real-world insulation applications.

Therefore, there is a need for improved systems and methods for detecting and characterizing polymer degradation during carbon tracking tests, which can (i) provide spatial and temporal resolution of degradation events, (ii) ensure consistency and control in the progression of failure mechanisms, and (iii) mitigate or account for the influence of air arcing, particularly at high test voltages exceeding 600 volts.

### SUMMARY

According to embodiments, systems, methods, and apparatuses to improve polymer degradation testing, especially in comparative electrical tracking evaluations, are provided. The embodiments include detection methods for identifying and characterizing polymer degradation through spatial, spectral, and temporal analysis of discharges and dry bands during tracking tests. The embodiments further provide dynamic monitoring and adjustment of testing parameters based on real-time comparison to reference degradation profiles, ensuring controlled and repeatable outcomes. Additionally, the embodiments encompass an apparatus configuration that employs inert gas atmospheres and coated electrodes, to effectively suppress air arcing and partial discharge events at higher test voltages, thereby facilitating accurate assessments of polymer insulation performance.

In embodiments, a method for identifying and locating polymer degradation during an electrical tracking test is provided. The method may include: detecting an air arcing or partial discharge in air surrounding a test specimen comprising a polymer; detecting a discharge occurring on a surface of the test specimen; detecting a location of a dry band formed in an initial stage of a carbon tracking operation taking place on the surface of the test specimen; generating, by a processor, a processing signal that temporally characterizes a sequence of discharge events corresponding to the carbon tracking operation; and correlating, by the processor, the processing signal with the discharge occurring on the surface of the test specimen to produce a correlation signal indicative of a temporal match between the processing signal and the carbon tracking operation.

In a further embodiment, a system of identifying and locating polymer degradation during an electrical tracking test is provided. The system may include: a detecting device configured to: detect an air arcing or partial discharge in air surrounding a test specimen comprising a polymer, detect a discharge occurring on a surface of the test specimen, and detect a location of a dry band formed in an initial stage of a carbon tracking operation taking place on the surface of the test specimen. The system may further include a computing device interface with the detecting device and configured to: generate a processing signal that temporally characterizes a sequence of discharge events corresponding to the carbon tracking operation, and correlate the processing signal with the discharge occurring on the surface of the test specimen to produce a correlation signal indicative of a temporal match between the processing signal and the carbon tracking operation.

### BRIEF DESCRIPTION OF FIGURES

FIG. 1 Illustrates a testing setup of a system, in accordance with the described embodiments.

FIG. 2 illustrates a testing apparatus, in accordance with the described embodiments.

FIG. 3A illustrates a detailed view of a set of electrodes, in accordance with the described embodiments.

FIG. 3B illustrates a detailed view of a set of electrodes, in accordance with the described embodiments.

FIG. 4 illustrates a method of identifying and locating polymer degradation during an electrical tracking test, in accordance with the described embodiments.

FIG. 5 illustrates a hardware diagram of an example detecting device, in accordance with the described embodiments.

### DETAILED DESCRIPTION

Systems and methods for identifying, controlling, and screening polymer degradation during electrical tracking and accelerated degradation tests are provided. More specifically, the systems and methods address limitations of existing technologies by introducing improved techniques for locating polymer degradation, controlling accelerated degradation processes for consistency across laboratories, and reducing air arcing at high voltages to improve polymer evaluation reliability. The embodiments discussed herein refer to the assessment of polymers, however it should be appreciated that the embodiments may apply to other materials such as (1) non-plastic insulating materials, such as fiberglass, mineral wool, cellulose, natural fibers, perlite, cementitious foam, and aerogel; (2) plastic-based insulating foams, including polystyrene, polyisocyanurate, polyurethane, and phenolic foam; and (3) general-purpose and engineering thermoplastics, such as polypropylene, polyethylene, polycarbonate, poly(phenylene ether), poly(ethylene terephthalate), poly(butylene terephthalate), poly(acrylonitrile-butylene-styrene), nylon 66 (polyamide 66), and nylon 6 (polyamide 6). Accordingly, in instances that refer to or discuss a “polymer”, it should be appreciated that these instances may also envision a general “insulating material” or material with insulating properties and/or with polymer-based ingredients.

Modern carbon tracking tests, such as those defined under UL 746A, IEC 60112, and ASTM D3638, rely on predetermined thresholds for leakage current to assess material resistance to carbon tracking. These tests monitor degradation progression indirectly, without spatial and temporal insight into key stages such as dry band formation, location-specific discharges, or corona/scintillation evolution. Additionally, these methods struggle with variability in identifying degradation mechanisms like tracking, erosion, or fire, leading to inconsistent results and limited diagnostic precision.

Systems and methods for detecting and locating polymer degradation during carbon tracking tests with enhanced spatial and temporal resolution are provided. The systems and methods may include the following functionalities: detecting air arcing/partial discharge in air surrounding the polymer specimen; detecting surface discharges on the test specimen, with analysis of emitted colors, wavelengths, sizes, and lengths corresponding to specific stages of degradation; identifying and locating dry bands during the initial stages of carbon tracking on the polymer surface; providing a process signal describing the sequence of degradation events on the polymer specimen; correlating the differential signals of discharges within a computing unit to yield a correlation signal, which may match the degradation timeline during the electrical tracking test. The systems and methods enable polymer degradation to be monitored and mapped to a database of known profiles for failure mechanisms (e.g., tracking, erosion, fire, and/or others). This database-guided functionality provides granular insight into degradation and allows real-time classification of failure modes.

These embodiments represent an improvement over existing technologies. In particular, the embodiments enable enhanced resolution, as real-time or near-real-time spatial and temporal tracking of degradation stages such as dry band formation, discharges, and failure mechanisms enhances diagnostic precision. Further, the embodiments support the classification of dominant degradation modes like tracking, erosion, and/or fire, which conventional techniques cannot distinguish. Additionally, by integrating discharge profiles into a process sequence, the embodiments yield richer data compared to leakage current-based assessments.

Accelerated degradation testing serves an essential role in screening and ranking polymer materials. Yet, according to ASTM D3638 and IEC 60112, the procedure for electrical tracking tests involves a fixed sequence from voltage setting to droplet application until failure occurs. This linear approach does not account for changing mechanisms or variations across batches of materials, often resulting in inconsistent outcomes either intralaboratory or interlaboratory. Factors such as temperature, humidity, oxygen exposure, UV radiation, and equipment parameters introduce significant variability in the degradation process, further complicating the ranking of materials.

Additional systems and methods provide an improved technique for controlling the accelerated degradation process of polymers to ensure consistent results across laboratories. The systems and methods may include the following functionalities: detecting degradation signals using a dedicated apparatus equipped with various sensors; comparing the detected signals to reference signals stored in a database to proactively classify the degradation type; and modifying equipment parameters (e.g., voltage, pressure, temperature, environmental conditions, and/or others) to achieve controlled degradation from initiation to completion. By dynamically monitoring and adjusting experimental conditions as degradation progresses, the systems and methods provide repeatable and predictable testing outcomes, regardless of interlaboratory or intralaboratory variations.

These embodiments represent an improvement over existing technologies. In particular, the embodiments ensure consistency across laboratories, as the dynamic adjustment of test parameters compensates for environmental and material variability, ensuring repeatability in accelerated degradation testing. Further, the embodiments enable direct control over the degradation process, thus reducing complications caused by mechanism shifts during testing. Additionally, the embodiments address poor repeatability in prior methods, especially for ranking materials over varied or accelerated testing scenarios.

Additionally, air arcing, particularly at test voltages exceeding 600 volts, presents challenges to existing carbon tracking tests. Existing attempts to mitigate this issue have involved rotating platinum electrodes 180 degrees, enabling measurements up to 1,000 volts AC and 1,000 volts DC. However, this method fails to prevent air arcing, resulting in inconsistent outcomes and measurements. Additionally, air arcing introduces a failure mode distinct from intended surface degradation mechanisms, compromising the relevance of test results for real-world applications.

Further, an improved apparatus and methods associated therewith are provided. In embodiments, the apparatus may include a sealed test chamber that may be configured with an inert gas purging system using gases such as helium, argon, or nitrogen, and electrodes coated with high dielectric constant materials, such as aluminum oxide (Al₂O₃), titanium dioxide (TiO₂), magnesium oxide (MgO), or calcium oxide (CaO). By controlling the gas environment and selectively coating the electrodes, this apparatus may reduces or eliminates unwanted air discharges, thus enabling accurate testing under conditions well above conventional voltage limits, typically beyond 600 volts and up to 2000 volts AC/DC. The improved apparatus thereby ensures that degradation pathways genuinely correspond to polymer surface degradation rather than extraneous air breakdown.

Compared to existing technologies, this embodiment addresses the longstanding challenge of air arcing, which has caused test inconsistencies, reduced accuracy, and limited maximum test voltages in conventional electrical tracking tests. Previous approaches, such as electrode repositioning, have proven insufficient because air breakdown still frequently occurs and compromises reliable material evaluation. By introducing inert gases into the testing environment and employing dielectric-coated electrodes, this embodiment may achieve uniform suppression of partial discharge events even at high test voltages, thus improving testing accuracy, repeatability, and consistency. Consequently, this embodiment facilitates precise measurements of polymer resistance to electrical degradation, enabling reliable, standardized evaluations and accurate comparative screening of polymeric materials under conditions more representative of real-world, high-voltage operating environments.

Embodiments for identifying and locating the degradation of polymer materials during electrical tracking tests are described. Conventional carbon tracking tests focus primarily on establishing a pass/fail threshold based on leakage current measurements (e.g., exceeding 0.5 or 1.0 amperes). However, these test fail to capture the dynamic progression of the degradation process, such as the formation of dry bands, corona discharge, scintillation discharge, and eventual carbonized tracking.

The described embodiments address these limitations by utilizing a detection technique, a processing signal framework, and real-time or near-real-time correlation with a database of known tracking mechanisms. The embodiments capture spatial, temporal, and spectral information of discharge events to classify failure modes such as tracking, erosion, or fire, thus enabling enhanced diagnostic precision for polymer screening and ranking applications.

An electrical tracking test is a standardized evaluation used to assess the resistance of insulating materials, such as polymers, to surface degradation when subjected to electrical stress in the presence of moisture and contamination. The test is designed to simulate real-world environmental and electrical conditions that can lead to the formation of conductive carbonized paths, or “tracks,” across the surface of the insulating material, potentially resulting in electrical failure or fire.

A typical electrical tracking test setup may include a test specimen, electrodes, a contaminant delivery mechanism, and an electrical power source. The test specimen may be a sample of the insulating material to be evaluated, generally fabricated into a flat and uniform surface. The specimen may be pre-conditioned to control for environmental factors such as temperature and humidity.

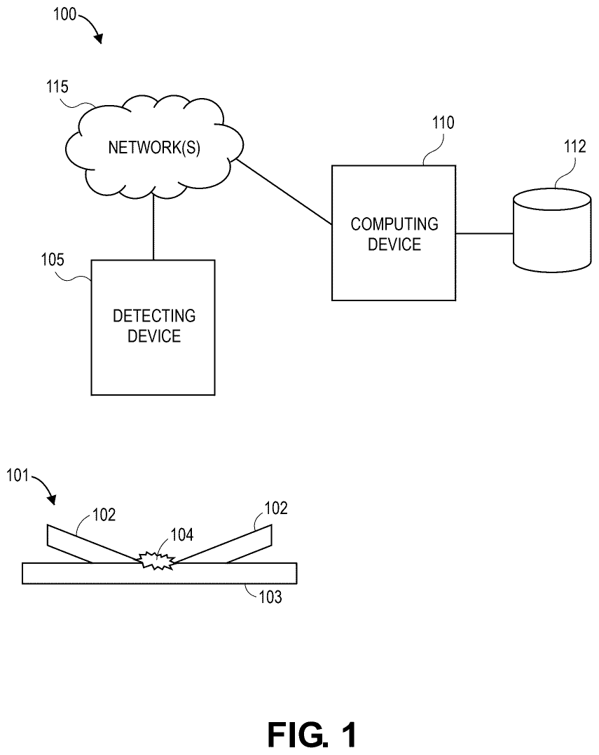

FIG. 1 illustrates a testing setup 101 of a system 100 according to the described embodiments. The system 100 may include a pair of electrodes 102 that may be positioned at an angle on the surface of a specimen 103. These electrodes 102 may be made of a corrosion-resistant conductive material such as platinum and may be spaced apart at a fixed distance (e.g., 4 mm). An alternating current (AC) voltage may be applied across the electrodes 102, thereby establishing an electric field along the surface of the specimen 103.

To simulate contamination, droplets of a standardized electrolyte solution may be periodically deposited on the surface of the specimen 103 between the electrodes 102. In the presence of the electric field and moisture, a discharge 104 may begin to form across the specimen 103 surface, as shown in FIG. 1, which may represent the onset of electrical tracking, where localized heating and material degradation begin to form a carbonized conductive path between the electrodes. The discharge 104, which may include corona discharges or scintillation arcs, may initiate localized surface degradation. The discharge 104 may also emit visible light (e.g., violet, red, or orange hues) and produce thermal energy, both of which contribute to the formation of conductive tracks.

Over time, if the surface degradation becomes significant, the leakage current between the electrodes may increase. A test failure may recorded when this current exceeds a predetermined threshold, such as 0.5 A or 1.0 A, in accordance with standards like IEC 60112 or ASTM D3638.

The outcome of the electrical tracking test may used to determine a comparative tracking index (CTI) of the material, which indicates the voltage level at which the material is likely to fail under specified conditions. However, conventional test methods do not provide sufficient insight into the location, evolution, or consistency of the degradation process, especially when failure mechanisms shift or when voltages exceed 600 V and introduce sustained air arcing phenomena.

In the initial stages of carbon degradation testing, partial discharges or air arcing may occur as localized bursts of energy between electrodes. These discharges can distort the intended carbon tracking evaluation and provide misleading test results. The system 100 may further include a detecting device 105 that may be capable of identifying various phenomena using, for example, digital imaging technology and a camera(s) such as a high-speed camera(s). The camera(s) may capture spatial data to locate the exact points of discharge in air and analyze their frequency, intensity, and trajectory across various voltages.

The detecting device 105 may include one or more digital cameras specifically selected to provide high-resolution imaging, spatial accuracy, and temporal resolution sufficient for detailed discharge event analysis. For example, the camera(s) may include a high-speed camera capable of recording video at frame rates of at least 1,000 frames per second (fps), preferably up to 10,000 fps or more, to accurately capture short-lived events such as air arcing and partial discharges. Further, the camera(s) may utilize specialized optical components, such as zoom lenses, macro lenses, or telecentric lenses, to closely image critical areas under test, thereby clearly resolving individual discharge points and patterns on or near the specimen 103. The camera(s) may additionally feature built-in spectral sensitivity or filters to detect and record differences in emitted wavelengths or colors, further facilitating identification of distinct phenomena such as corona discharge (typically violet-colored), scintillation discharge (orange-red), or high-temperature carbonized arcs (white).

The system 100 may be configured to differentiate air arcing from surface discharges using spectral analysis, including analyzing variations in wavelengths emitted during discharges. Air arcing is characterized by distinct wavelengths missing key signatures of surface degradation, enabling the system 100 to distinguish failure mechanisms during the test. This differentiation may help ensure that surface degradation is measured accurately without interference from unintended air arcing phenomena.

Surface discharges, which may occur on the specimen 103 itself, are relevant to the carbon tracking process. The digital camera as part of the detecting device 105 may employ spectroscopic capabilities to detect and locate these discharges in real-time or near-real-time. The system 100 may further include a computing device 110 that may map the distribution of discharge events, and capture detailed spatial and temporal data to determine their correlation with material breakdown.

The digital camera(s) may be interfaced with the computing device 110 through a hardwired connection or a network connection 115, thus enabling seamless data transfer and real-time processing of captured images and video. In particular, the digital camera(s) may connect to the computing device 110 via a Universal Serial Bus (USB), High-Definition Multimedia Interface (HDMI), or Ethernet cable, such as depending on the required data transfer speed and digital camera specifications. Alternatively, the digital camera(s) may interface with the computing device 110 over a local area network (LAN) or wireless network (Wi-Fi), or other network connection. In particular, network-enabled cameras may equipped with Ethernet ports or built-in wireless capabilities that may communicate with the computing device 110 without the need for a direct physical connection.

Surface discharges may evolve through distinct stages, beginning with violet corona discharge, followed by red/orange scintillation discharge, and eventually leading to white-hot arcs as the material carbonizes. The embodiments identify each stage through the emitted color, wavelength (e.g., using a spectrometer), length, and/or intensity of the discharge. The computing device 110 may record and spatially tag these signals to identify a degradation sequence and pinpoint areas where material resistance is progressively weakening. For example, the computing device 110 may pinpoint dry bands formed by Joule heating or localized energy bursts near the electrodes 102 to assess critical points of failure.

Dry bands may represent an early precursor to material failure during carbon tracking. These bands may arise due to Joule heating on the specimen 103 surface, which may lead to localized areas of electrical insulation breakdown. The detecting device 105 and related components may capture the formation and location of dry bands in real time. By analyzing discharge patterns and the temperature gradient across the surface, the computing device 110 may track where these dry bands emerge, whether near the center of the electrodes 102 or at their edges.

The system may incorporate a set of optical and/or infrared sensors to measure the thermal energy associated with dry band formation. This data may provide a set of insights into the progression of degradation and enable the computing device 110 to predict failure scenarios based on dry band dynamics. Identifying dry bands early may enable for a more comprehensive understanding of the carbon tracking process and may enable preventative action during material development.

To characterize the dynamic process of carbon tracking, the embodiments may provide a processing signal that maps the sequence of degradation events over time. Signals generated by air arcing, surface discharges, and dry bands may be collected and fed into the computing device 110, which may synthesize these differential process signals and generate a temporal map of the degradation sequence. For example, a signal might record the progression from corona discharge (violet) to scintillation discharge (red/orange), followed by prolonged surface tracking and eventual material failure.

The temporal mapping feature may enable individuals to pinpoint specific moments of degradation progression and correlate them to environmental factors, voltage amplifications, and/or droplet application rates. By visualizing the degradation timeline, the embodiments may aid in understanding how different polymer materials respond to carbon tracking conditions and provide actionable data for improving insulation materials.

The computing device 110 may interface with a database 112 that may store reference profiles for known degradation mechanisms. The computing device 110 may match the signals collected during the test, including spatial data, temporal data, and/or spectral information, against reference signals in the database 112 to classify a dominant failure mechanism.

For instance, the computing device 110 may determine whether the observed degradation is primarily tracking-related, erosion-driven, or fire-induced based on correlations with preexisting profiles. Further, surface discharges emitting red/orange wavelengths of a specific intensity might align with a tracking-dominated mechanism, while prolonged white-hot discharges might indicate erosion or combustion.

The computing device 110 may assess the relationship between discharge color, wavelength, and the degradation stage. Using certain techniques (e.g., spectroscopic methods), the computing device 110 may collect data on emitted wavelengths during discharges, and may note transitions from violet corona discharge to red/orange scintillation discharge, thus aiding in the understanding of material resilience and degradation patterns.

Furthermore, the computing device 110 may integrate spatial measurements, detecting the horizontal and vertical lengths of carbon tracking paths. The ability to measure and map these paths may provide a quantifiable metric for material sensitivity to electrical stresses. In particular, longer tracking paths might signify weaker insulation properties, while shorter paths may indicate higher material resistance under the same test conditions.

Using certain data from discharges, the computing device 110 may measure a physical length of carbon tracking paths in real-time or near-real-time. Certain parameters, such as the width and density of tracked regions, may be analyzed to predict when material breakdown will occur. This geographical tracking capability may enable insights into degradation mechanics, allowing researchers to design more resistant polymer products.

In addition, the computing device 110 may identify the shape of the discharge pathways (e.g., linear, branched, or chaotic) to further differentiate failure mechanisms. The combined spatial, spectral, and temporal analysis may help ensure that polymer degradation is diagnosed with greater accuracy than traditional pass/fail leakage current tests.

Embodiments for conducting polymer degradation tests, addressing the variability and inconsistency inherent in traditional degradation testing approaches, whether conducted intralaboratory or interlaboratory, are provided. The embodiments utilize a combination of a detecting device (e.g., the detecting device 105) and a computing device (e.g., the computing device 110) to monitor, analyze, and control the degradation process from its initiation to completion. Signals generated during degradation may be compared to reference signals stored in a database (e.g., the database 112), and equipment parameters may be dynamically adjusted to ensure that failure mechanisms, including tracking, erosion, fire, and/or others, progress as anticipated.

While polymer degradation techniques such as electrical tracking, thermal degradation, photo-oxidation, hydrolysis, biodegradation, and chemical degradation often result from complex interplay between environmental factors, test setup, and material characteristics, the present embodiments seek to deliver repeatable results independent of varying conditions. By introducing a feedback mechanism that modifies equipment parameters and enables iterative corrective setups, the embodiments enhance and improve the utility of accelerated degradation testing to reliably rank or validate polymeric materials for specific applications.

According to embodiments, the detecting device 105 may be employed to capture a set of degradation signals generated throughout a test of a polymer material (e.g., the specimen 103). These signals may represent phenomena such as electrical discharges, thermal propagation, UV responses, and breakdown processes occurring within the tested polymer material. As discussed herein, a digital camera (e.g., a high-resolution digital camera) may serve as a sensory tool, and may be configured to capture spatial, spectral, and temporal data related to degradation processes.

For degradation processes like electrical tracking, the camera may record location-specific discharges and spatial trends, and provide any resulting data to the computing device 110 for further processing. In certain embodiments, the computing device 110 may map surface discharges as they propagate across the specimen 103, while spectral analysis may enable the identification of colors and wavelengths emitted by degradation events, such as violet corona discharge or red scintillation discharge. For thermal degradation or hydrolysis, the detecting device 105 may identify heat signatures or localized polymer interactions, such as those triggered by environmental stressors such as elevated temperatures or moisture exposure.

The computing device 110 may compare any signals captured during testing to reference signals stored in the database 112. These reference signals may correspond to known degradation profiles, which may indicate expected material behavior under specific stress conditions. For example, in electrical tracking tests, expected discharge paths, failure patterns, and dominant mechanisms (e.g., surface tracking or air arcing) may be encoded as reference signals within the database 112.

The computing device 110 may facilitate the comparing dynamically and in real-time or near-real-time, and identify deviations between the observed signal and the reference signal. If any deviations are detected (e.g., if discharges occur in unintended areas such as between electrodes rather than on the polymer surface), the computing device 110 may flag the degradation test for a set of corrective actions. The reference comparison may enable accurate measurement of how closely the degradation process aligns with intended outcomes.

The embodiments may further support a feedback mechanism that may modify one or more equipment parameters based on any detected signals and their alignment (or misalignment) with reference signals. This feedback may enable real-time or near-real-time control over the failure mechanism progression. For example, if premature air arcing between electrodes is detected, one or more equipment parameters may be adjusted to suppress unintended discharge, such as modifying electrode positioning, droplet mass, voltage levels, and/or environmental conditions like pressure or humidity. For further example, if test results deviate from the expected degradation profile, the test procedure and/or equipment setup may be automatically reset to re-align the process with the reference signal. The ability to dynamically regulate equipment parameters may mitigate any inconsistencies caused by varying test setups, environmental conditions, and/or material batches, thus helping facilitate reproducibility of results across different laboratories and iterations.

In aspects, the embodiments may enable screening and/or ranking of various polymeric materials under degradation tests. For example, in electrical tracking tests, any discharge locations, propagation lengths, and/or wavelengths observed during testing may be compared to reference signals across different material types. If detected signals match the reference signal during the initial phase (e.g., first five to ten runs), the test may proceed uninterrupted. However, if detected signals deviate, such as detecting air arcing between electrodes, the system may reset the test setup until parameters align with the reference signal. These capabilities may help ensure that materials are evaluated consistently and ranked accurately based on their degradation performance. Further, the ability to identify and control deviations early in the test may reduce variability among different batches and support reliable evaluation of polymer properties.

The embodiments may further validate the performance of single polymeric materials, such as polycarbonate, under certain targeted degradation conditions. For example, during electrical tracking tests, surface discharge emission (e.g., colors, wavelengths, and/or progression paths) may be captured and compared to reference signals. The detecting device 105 may record the evolution of these discharges throughout the test, thus revealing how the material undergoes failure mechanisms like tracking, erosion, and/or fire.

The computing device 110 may analyze temporal data to reveal relationships between these mechanisms (e.g., how tracking transitions into erosion or how erosion evolves into fire). By validating certain performance metrics against certain known degradation profiles, the embodiments may provide actionable insights into the reliability and durability of single-material types under real-world stress conditions.

From the beginning of the test to its completion, the embodiments may record data detailing the progression of any degradation processes, and may correlate the observed signals with preexisting failure mechanism profiles. The recorded data may highlight any material responses to varying stress conditions, such as higher voltages, increased temperatures, and/or prolonged UV exposure.

The computing device 110 may compile and analyze this recorded data to establish relationships among failure mechanisms. For example, the computing device 110 may determine how the degradation pathway for a particular polymer moves from surface tracking, to secondary erosion, to combustion. This analytical feature not only supports ranking and screening applications but also advances the scientific understanding of polymer degradation cascades under different environmental stressors.

The embodiments address concerns related to variability among test results within or across laboratories. Traditional methods often fail to account for factors that shift failure mechanisms mid-test, such as added heat or changing environmental variables. The embodiments mitigate these inconsistencies by introducing a systematic process of detecting, comparing, and adjusting test parameters as needed. By aligning observed signals with reference signals throughout the test, the embodiments help ensure control over the degradation process, thus enabling consistent and repeatable outcomes, regardless of laboratory setting.

An apparatus, system, and device designed to eliminate air arcing and partial discharge during testing at high voltages (e.g., beyond 600 volts) is provided. The embodiments may include: a purging system to introduce inert gases, such as helium, argon, and nitrogen, into the testing chamber. These gases may reduce the mean free path of electrons to prevent air arcing, as mean free path is inversely proportional to pressure. The embodiments may further include a set of coated electrodes that may be made from high dielectric constant materials, including Al₂O₃, TiO₂, MgO, and CaO, such as to suppress the inception of air arcing/discharge in atmospheric air; and a set of environmental control features within the chamber which may prevent unintended degradation mechanisms while maintaining the desired breakdown via dissociative electron attachment, which may disrupt polymeric bonds for material screening. These embodiments may enable consistent and reliable evaluation of polymer degradation, especially for insulation rankings under high-voltage applications.

These embodiments represent an improvement over existing technologies. In particular, the embodiments enable the elimination of air arcing, as inert gas purging and dielectric-coated electrodes suppress air arcing entirely, ensuring intended surface degradation evaluation. Further, the embodiments enable accurate and repeatable testing beyond 600 volts without introducing failure modes unrelated to the surface tracking of the tested polymer. Additionally, by focusing on polymer breakdown via dissociative electron attachment, the embodiments provide meaningful rankings for insulating materials under high-stress conditions.

According to embodiments, a system, device, and apparatus for testing the degradation of polymeric insulating materials during electrical tracking evaluations, particularly at elevated test voltages exceeding 600 volts, are provided. These embodiments aim to address key challenges, such as the occurrence of air arcing and partial discharges, that compromise the accuracy and reliability of conventional tracking tests. The embodiments achieve this by leveraging a controlled testing chamber integrated with systems for droplet application, gas purging, pressure regulation, and specially coated electrodes. The embodiments enable high-fidelity testing by eliminating unwanted arcing phenomena, allowing for accurate assessment of polymer degradation mechanisms under a range of electrical and environmental conditions. The embodiments also enable dissociative electron attachment at controlled settings to deliberately disrupt polymeric bonds, thus ensuring repeatable and precise material ranking and screening.

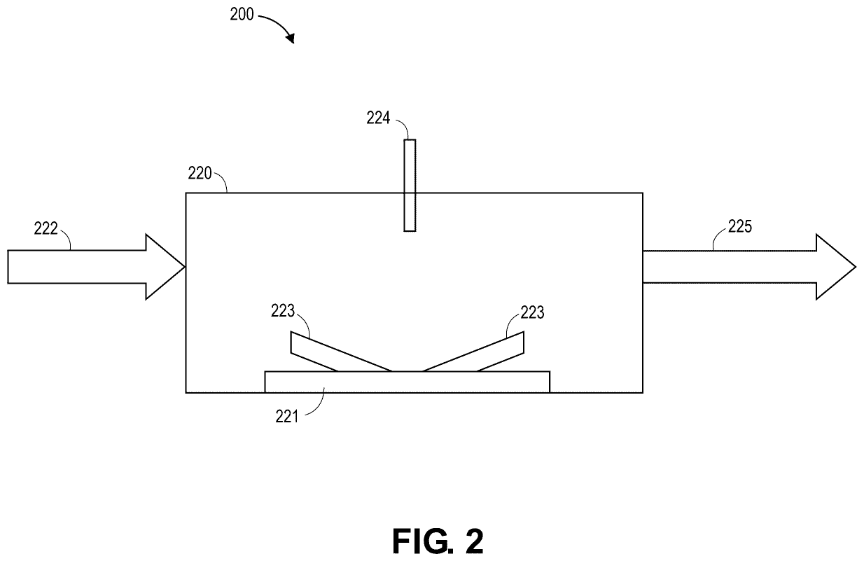

FIG. 2 illustrates an apparatus 200 according to various embodiments. The apparatus 200 may include a testing chamber 220 that may serve as an enclosed environment for performing one or more electrical tracking tests. The chamber 220 may be equipped with a purging system, including a gas purge inlet 222 and a gas pump outlet 225, that may introduce inert gases such as helium, argon, or nitrogen to regulate the internal atmosphere. By purging inert gases into the chamber 220 via the gas purge inlet 222, the mean free path of electrons may be reduced, thereby suppressing air arcing and partial discharge.

Pressure within the chamber 220 may be controlled to specific levels depending on any test requirement(s). For example, in certain embodiments, pressure may range from 14.7 psi (atmospheric pressure) up to 88.2 psi for specific tests. In other embodiments, the chamber 220 may be configured for low-pressure tests, for example with pressures ranging from 5 psi to 14.7 psi. This variability in pressure control may ensure flexibility in test conditions and enable for experimentation across a wide range of polymer materials.

The pressure regulation features may manage the electron interactions associated with air breakdown. In particular, as the mean free path of electrons is inversely proportional to the pressure, the regulated chamber environment may ensure that air arcing is suppressed even under high voltages, thus enabling reliable surface degradation assessments instead of unintended arcing phenomena.

The apparatus 200 may include set of a droplet application components for introducing, via a dropping device 224, a set of standardized electrolyte droplets onto the surface of a polymer specimen 221 during testing. The system may enable for controlled droplet mass, for example ranging from 20 mg to 120 mg, and may ensure uniform application rates. The control over droplet application may minimize variability in the testing process, helping ensure that the degradation occurs uniformly across test specimens.

The sequence and intervals for the droplets may be customizable, and the set of droplet application components may operate seamlessly under the controlled atmosphere of the chamber 220. Whether at low pressure, high pressure, or standard atmospheric settings, the set of droplet application components may ensure consistent electrolyte application, aiding in the detection and analysis of carbon tracking progression.

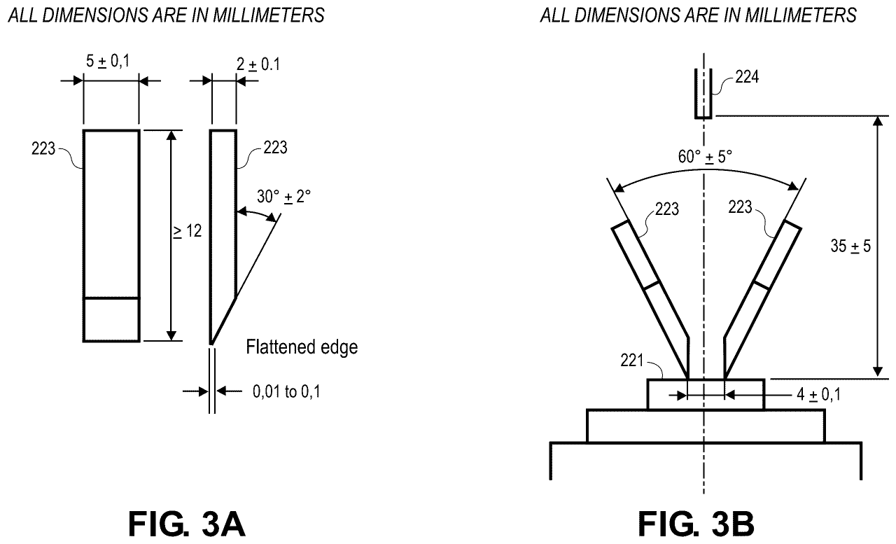

The apparatus 200 may further include a set of electrodes 223 (e.g., platinum electrodes) that may be designed with a rectangular cross-section, for example measuring 5.0 mm×2.0 mm (or other dimensions) and polished to a 30° (or other degrees) chisel-point edge. The set of electrodes 223 may be polished using sandpaper (e.g. sandpaper #3000) to ensure consistency in edge smoothness, which may enhance the uniformity of electrical discharge interactions between the set of electrodes 223 and the polymer specimen 221. The design of the set of electrodes 223 may include a fixed extension, for example an extension of 20 mm (or other dimensions), and adjustable electrode spacing, for example ranging from 4 mm to 12 mm (or other dimensions or ranges of dimensions). The set of electrodes 223 and design thereof are illustrated in greater detail in FIGS. 3A and 3B.

In an embodiment, the set of electrodes 223 may be coated with a material(s) having a high dielectric constant, such as aluminum oxide (Al₂O₃), titanium dioxide (TiO₂), magnesium oxide (MgO), or calcium oxide (CaO). These coatings may be applied to suppress the inception of air arcing and partial discharge under atmospheric air conditions. The high dielectric constant of the coated materials may enable better control over the electric field distribution between the set of electrodes 223, which may protect the integrity of the surface-tracking test.

The coated electrodes 223 may operate within a higher voltage range, for example 600 to 2000 volts, either AC or DC. By eliminating premature discharges and focusing the energy on the polymer surface, these coated electrodes 223 may enable for more accurate testing and reproducible results, even for high-voltage applications.

The apparatus 200 may incorporate the use of inert gases, such as helium, argon, or nitrogen, that may be purged into the chamber 220 to modify the testing environment. These gases may help reduce the likelihood and severity of air arcing by adjusting the mean free path of electrons. This is particularly advantageous for tests conducted at elevated voltages, such as ranges from 600 volts to 2000 volts, where unwanted partial discharge may interfere with degradation results.

In cases where helium is introduced into the chamber 220, its low atomic mass and high ionization potential make it especially effective for minimizing arcing. For certain polymer applications where pressure and voltage sensitivity are critical, the inclusion of helium may provide improved control over the test environment. This feature of the apparatus 200 may ensure that the degradation is caused solely by surface-tracking phenomena and not by uncontrolled air breakdown.

The apparatus 200 may be designed to initiate dissociative electron attachment, a process that may disrupt polymeric bonds in localized areas. Under a controlled electric field and droplet application, dissociative attachment may lead to progressive insulation degradation, which may eventually result in carbonized tracking or other forms of breakdown. This intentional bond disruption may ensure that polymers can be effectively screened and ranked for their electrical performance and durability.

By enabling precise adjustments to electric field strength, pressure, and droplet application, the apparatus 200 may optimize the degradation process to match real-world conditions for polymer insulation performance. The ability to control these variables may enhance repeatability, even in accelerated tests, thereby improving interlaboratory and intralaboratory consistency.

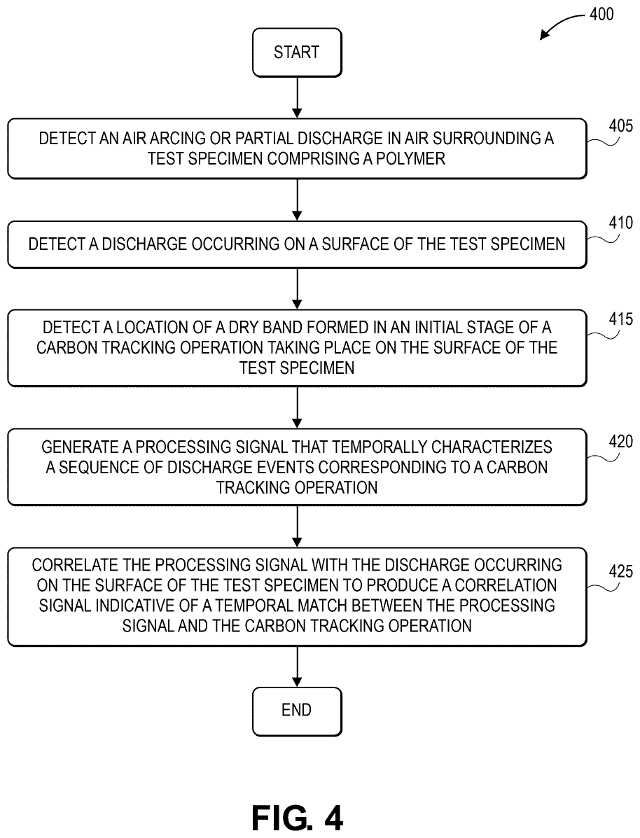

FIG. 4 illustrates a method 400 of identifying and locating polymer degradation during an electrical tracking test. According to embodiments, the method 400 may be performed by a combination of a detecting device (e.g., the detecting device 105 as described with respect to FIG. 1) and a computing device (e.g., the computing device 110 as described with respect to FIG. 1).

The method 400 may begin at block 405 at which the detecting device may detect an air arcing or partial discharge in air surrounding a test specimen comprising a polymer. At block 410, the detecting device may detect a discharge occurring on a surface of the test specimen, such as a via a digital camera capturing a set of digital images depicting the surface of the test specimen.

At block 415, the detecting device may detect a location of a dry band formed in an initial stage of a carbon tracking operation taking place on the surface of the test specimen. In embodiments, the detecting device may detect a wavelength of electromagnetic radiation emitted by the discharge occurring on the surface of the test specimen.

In embodiments, a computing device may analyze the set of digital images to determine a location of the discharge occurring on the surface of the test specimen relative to at least one electrode positioned on the surface. Further, the computing device may analyze the set of digital images to determine at least one color of the discharge occurring on the surface of the test specimen, and correlate the at least one color with a stage of the carbon tracking operation. Alternatively or additionally, the computing device may analyze the set of digital images to measure a length of a carbon tracking path formed during the carbon tracking operation taking place on the surface of the test specimen.

At block 420, the computing device may generate a processing signal that temporally characterizes a sequence of discharge events corresponding to the carbon tracking operation. Further, at block 425, the computing device may correlate the processing signal with the discharge occurring on the surface of the test specimen to produce a correlation signal indicative of a temporal match between the processing signal and the carbon tracking operation.

In embodiments, the computing device may compare the correlation signal to a set of reference signals stored in a database to determine a failure mechanism associated with polymer degradation, where the failure mechanism may be one of carbon tracking, erosion, or combustion. Further, the computing device may dynamically adjust, based on the correlation signal, one or more parameters of the electrical tracking test to control a progression of the carbon tracking operation.

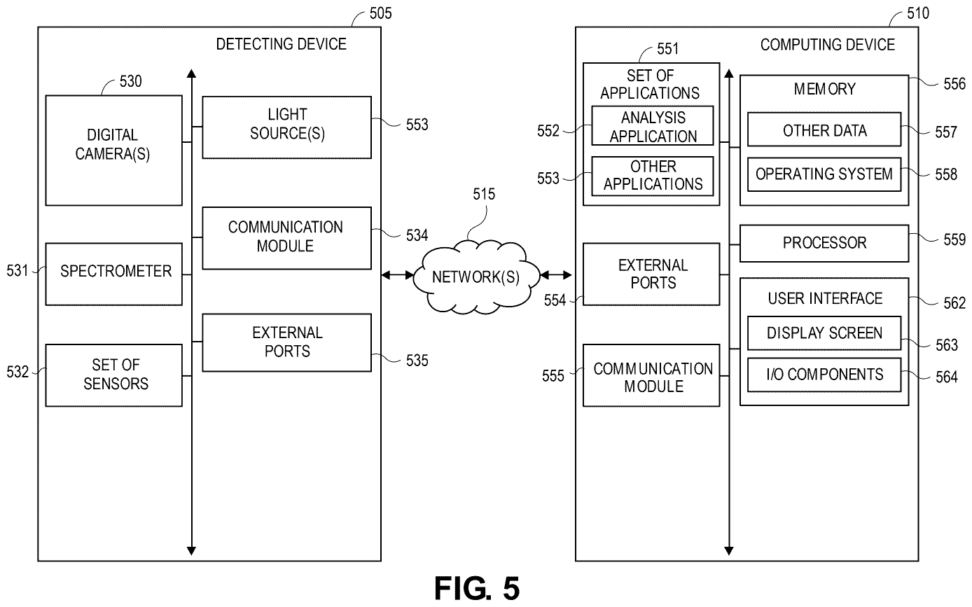

FIG. 5 illustrates a hardware diagram of an example detecting device (e.g., the detecting device 505 as described with respect to FIG. 1) and example computing device 510 (e.g., the computing device 510 as described with respect to FIG. 1), in which the functionalities as discussed herein may be implemented. It should be appreciated that the components of the devices 505, 510 are merely exemplary, and that additional or alternative components and arrangements thereof are envisioned. Generally, the detecting device 505 and the computing device 510 may interface with each other via a network(s) 515, as discussed herein.

As illustrated in FIG. 5, the detecting device 505 may include one or more digital cameras 530 that may be configured to capture digital images and/or videos for spatial, temporal, and spectral characterization of discharge events occurring on or around a polymer specimen under test. The detecting device 505 may further incorporate a spectrometer 531 that may detect and analyze wavelength emissions from discharge phenomena, thus enabling accurate classification of polymer degradation stages based on spectral signatures.

Additionally, the detecting device 505 may include a set of sensors (e.g., optical, thermal (infrared), and/or environmental sensors) that may be included to measure supplemental properties such as local temperature variation, humidity, or intensity of discharge events, providing complementary data to support comprehensive degradation analysis. One or more dedicated light sources 533 may be integrated into the detecting device 505 to ensure sufficient and uniform illumination during imaging, especially under low-ambient-light conditions.

A communication module 534 capable of wired connectivity and/or wireless connectivity may be provided to enable transfer of data between the detecting device 505 and the computing device 510 in real-time or near-real-time. Additionally, the detecting device may be equipped with a set of external ports 535, including Ethernet, USB, HDMI, or other standard connectors, to enable integration with peripheral devices, computing units, controllers, or network infrastructure.

The computing device 510 may include a processor 559 as well as a memory 556. The memory 556 may store an operating system 558 capable of facilitating the functionalities as discussed herein as well as a set of applications 551 (i.e., machine readable instructions). For example, one of the set of applications 551 may be an analysis application 552 that may be configured to facilitate the data analysis functionalities as discussed herein. It should be appreciated that one or more other applications 553 are envisioned.

The processor 559 may interface with the memory 556 to execute the operating system 558 and the set of applications 551. According to some embodiments, the memory 556 may also store other data 557. The memory 556 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The computing device 510 may further include a communication module 555 configured to communicate data via the network(s) 515. According to some embodiments, the communication module 555 may include one or more transceivers (e.g., WAN, WWAN, WLAN, and/or WPAN transceivers) functioning in accordance with IEEE standards, 3GPP standards, or other standards, and configured to receive and transmit data via one or more external ports 554.

The computing device 510 may further include a user interface 562 configured to present information to a user and/or receive inputs from the user. As shown in FIG. 5, the user interface 562 may include a display screen 563 and I/O components 564 (e.g., ports, capacitive or resistive touch sensitive input panels, keys, buttons, lights, LEDs, external or built in keyboard). According to some embodiments, the user may access the computing device 510 via the user interface 562 to review information, make selections, and/or perform other functions.

In some embodiments, the computing device 510 may perform the functionalities as discussed herein as part of a “cloud” network or may otherwise communicate with other hardware or software components within the cloud to send, retrieve, or otherwise analyze data.

In general, a computer program product in accordance with an embodiment may include a computer usable storage medium (e.g., standard random access memory (RAM), an optical disc, a universal serial bus (USB) drive, or the like) having computer-readable program code embodied therein, wherein the computer-readable program code may be adapted to be executed by the processor 559 (e.g., working in connection with the operating system 558) to facilitate the functions as described herein. In this regard, the program code may be implemented in any desired language, and may be implemented as machine code, assembly code, byte code, interpretable source code or the like (e.g., via Golang, Python, Scala, C, C++, Java, Actionscript, Objective-C, Javascript, CSS, XML). In some embodiments, the computer program product may be part of a cloud network of resources.

In embodiments, a method for dynamically controlling test parameters during a polymer degradation test is provided. The method may include: detecting, by a detecting component, a degradation indicator associated with polymer degradation during the polymer degradation test; comparing, by a processor, the degradation indicator to at least one stored reference parameter representing an expected polymer degradation condition; and modifying, by the processor based on the comparing, at least one test parameter of the polymer degradation test, to actively maintain or achieve a predetermined degradation mechanism throughout the polymer degradation test.

In embodiments, the degradation indicator may correspond to a characteristic selected from one of: an electrical discharge, a thermal emission, an optical emission, a wavelength, a shape, and a color.

In embodiments, the at least one test parameter of the polymer degradation test may be one of: an applied voltage, an electrode spacing, a droplet application rate, an environmental temperature, an ambient pressure, and a humidity.

In embodiments, modifying the at least one test parameter may include adjusting the at least one test parameter in real-time during the polymer degradation test to maintain continuous alignment with the at least one stored reference parameter.

In embodiments, the polymer degradation test may include an electrical tracking test, and the degradation indicator may correspond to characteristics of discharge events occurring on or near a surface of a polymer-based specimen used in the electrical tracking test.

In embodiments, the detecting component may include at least one digital camera configured to capture a set of digital images. Further, the processor may analyze the set of digital images to measure at least one of: a length, a width, a shape, or a propagation path of carbon tracking formed on the polymer-based specimen.

In embodiments, the processor may determine whether a detected discharge event matches an expected discharge event defined by the at least one stored reference parameter, and may automatically reset the polymer degradation test when the detected discharge event fails to match the expected discharge event.

In a further embodiment, an apparatus for reducing or eliminating air arcing and partial discharge events during an electrical tracking test of a polymer specimen may be provided. The apparatus may include: a chamber configured to enclose the polymer specimen and a set of electrodes during the electrical tracking test, wherein each electrode of the set of electrodes is coated with a dielectric material; a dropping component configured to apply a set of droplets of electrolyte solution onto a surface of the polymer specimen; a set of pumping components configured to regulate pressure and gas flow within the chamber; and a set of purging components configured to introduce an inert gas into the chamber.

Although the following text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that may be permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that may be temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it may be communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment, or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical.

What is claimed is:

1. A method for identifying and locating polymer degradation during an electrical tracking test, the method comprising:
detecting an air arcing or partial discharge in air surrounding a test specimen comprising a polymer;
detecting a discharge occurring on a surface of the test specimen;
detecting a location of a dry band formed in an initial stage of a carbon tracking operation taking place on the surface of the test specimen;
generating, by a processor, a processing signal that temporally characterizes a sequence of discharge events corresponding to the carbon tracking operation; and
correlating, by the processor, the processing signal with the discharge occurring on the surface of the test specimen to produce a correlation signal indicative of a temporal match between the processing signal and the carbon tracking operation.

2. The method of claim 1, wherein detecting the discharge occurring on the surface of the test specimen comprises:
capturing a set of digital images depicting the surface of the test specimen using at least one digital camera.

3. The method of claim 2, further comprising:
analyzing, by the processor, the set of digital images to determine a location of the discharge occurring on the surface of the test specimen relative to at least one electrode positioned on the surface.

4. The method of claim 2, further comprising:
analyzing, by the processor, the set of digital images to determine at least one color of the discharge occurring on the surface of the test specimen.

5. The method of claim 4, further comprising:
correlating, by the processor, the at least one color with a stage of the carbon tracking operation.

6. The method of claim 2, further comprising:
analyzing, by the processor, the set of digital images to measure a length of a carbon tracking path formed during the carbon tracking operation taking place on the surface of the test specimen.

7. The method of claim 1, further comprising:
detecting a wavelength of electromagnetic radiation emitted by the discharge occurring on the surface of the test specimen.

8. The method of claim 1, further comprising:
comparing the correlation signal to a set of reference signals stored in a database to determine a failure mechanism associated with polymer degradation.

9. The method of claim 8, wherein comparing the correlation signal to the set of reference signals comprises:
comparing the correlation signal to the set of reference signals to determine the failure mechanism associated with polymer degradation, wherein the failure mechanism is one of carbon tracking, erosion, or combustion.

10. The method of claim 1, further comprising:
dynamically adjusting, by the processor based on the correlation signal, one or more parameters of the electrical tracking test to control a progression of the carbon tracking operation.

11. A system of identifying and locating polymer degradation during an electrical tracking test, comprising:
a detecting device configured to:
detect an air arcing or partial discharge in air surrounding a test specimen comprising a polymer,
detect a discharge occurring on a surface of the test specimen, and
detect a location of a dry band formed in an initial stage of a carbon tracking operation taking place on the surface of the test specimen;
a computing device interface with the detecting device and configured to:
generate a processing signal that temporally characterizes a sequence of discharge events corresponding to the carbon tracking operation, and
correlate the processing signal with the discharge occurring on the surface of the test specimen to produce a correlation signal indicative of a temporal match between the processing signal and the carbon tracking operation.

12. The system of claim 11, wherein to detect the discharge occurring on the surface of the test specimen, the detecting device is configured to:
capture, by a digital camera, a set of digital images depicting the surface of the test specimen using at least one digital camera.

13. The system of claim 12, wherein the computing device is further configured to:
analyze the set of digital images to determine a location of the discharge occurring on the surface of the test specimen relative to at least one electrode positioned on the surface.

14. The system of claim 12, wherein the computing device is further configured to:
analyze the set of digital images to determine at least one color of the discharge occurring on the surface of the test specimen.

15. The system of claim 14, wherein the computing device is further configured to:
correlate the at least one color with a stage of the carbon tracking operation.

16. The system of claim 12, wherein the computing device is further configured to:
analyze the set of digital images to measure a length of a carbon tracking path formed during the carbon tracking operation taking place on the surface of the test specimen.

17. The system of claim 11, wherein the detecting device is further configured to:
detect a wavelength of electromagnetic radiation emitted by the discharge occurring on the surface of the test specimen.

18. The system of claim 11, wherein the computing device is further configured to:
compare the correlation signal to a set of reference signals stored in a database to determine a failure mechanism associated with polymer degradation.

19. The system of claim 18, wherein the failure mechanism associated with the polymer degradation is one of carbon tracking, erosion, or combustion.

20. The system of claim 11, wherein the computing device is further configured to:
dynamically adjust, based on the correlation signal, one or more parameters of the electrical tracking test to control a progression of the carbon tracking operation.
